(19) United States
(12) Patent Application Publication
(10) Pub. No.: US 2026/0137274 A1
(43) Pub. Date: May 21, 2026

(54) ANALYZING THE BLINK BEHAVIOR OF AN EYE

(71) Applicant: Alcon Inc., Fribourg (CH)

(72) Inventors: Martin Gruendig, Rangsdorf (DE); Armin Haupt, Berlin (DE)

(21) Appl. No.: 19/381,297

(22) Filed: Nov. 6, 2025

Related U.S. Application Data
(60) Provisional application No. 63/722,666, filed on Nov. 20, 2024.

(51) Int. Cl. A61B 3/14; A61B 3/113

(52) U.S. Cl. CPC A61B 3/14; A61B 3/113

(57) ABSTRACT

In certain embodiments, an ophthalmic system includes a camera system and a computer. The camera system obtains eye images of an eye (that has an eyelid) during an imaging period. The computer system receives the eye images of the eye from the camera system and extracts a column image from each eye image of the eye images to yield column images. Each column image has a respective longitudinal axis substantially parallel to a vertical axis of the eye. Each column image shows at least the eyelid of the eye at a respective time during the imaging period. The computer system generates a time sequence image from the column images. The time sequence image comprises the column images in temporal order and indicates the position of the eyelid at respective times during the imaging period. The computer system outputs a blink analysis output that includes the time sequence image.

### FIELD

Embodiments of the present disclosure relate to analyzing the blink behavior of an eye.

### BACKGROUND

Analyzing the blink behavior of an eye may involve, e.g., determining the rate of blinks and the completeness of blinks performed by the eye. Current approaches to analyzing blink behavior typically involve recording a video of the eye and then analyzing the video. Medical personnel may inspect the video or image processing may be applied to the video to determine the blink rate and the completeness of blinks.

### SUMMARY

In certain embodiments, an ophthalmic system includes a camera system and a computer. The camera system obtains eye images of an eye (that has an eyelid) during an imaging period. The computer system receives the eye images of the eye from the camera system and extracts a column image from each eye image of the eye images to yield column images. Each column image has a respective longitudinal axis substantially parallel to a vertical axis of the eye. Each column image shows at least the eyelid of the eye at a respective time during the imaging period. The computer system generates a time sequence image from the column images. The time sequence image comprises the column images in temporal order and indicates the position of the eyelid during the imaging period. The computer system outputs a blink analysis output that includes the time sequence image.

Embodiments may include one, more than one, any combination of, or all of the following:

The computer system extracts the column image from each eye image by detecting a central region of the eye in at least one eye image and extracting the column image from the eye image, where the column image includes at least a portion of the central region.

The computer system detects the position of the eyelid in the time sequence image. The computer system may detect the position of the eyelid by detecting an eyelid marker of the eyelid in the time sequence image, where the eyelid marker indicates the eyelid, and detecting the position of the eyelid according to the eyelid marker. The computer system may detect the position of the eyelid by detecting the pupil of the eye in the time sequence image, detecting an upper border between the pupil and the eyelid, and detecting the position of the eyelid according to the upper border between the pupil and the eyelid.

The computer system determines an open position of the eyelid in the time sequence image, where the open position of the eyelid indicates the eye is open. The computer system may provide an open eyelid overlay superimposed onto the open position of the eyelid in the time sequence image.

The computer system determines a closed position of the eyelid in the time sequence image, where the closed position of the eyelid indicates the eye is closed. The computer system may provide a closed eyelid overlay superimposed onto the closed position of the eyelid in the time sequence image.

The computer system identifies one or more eye blinks in the time sequence image. The computer system may identify an eye blink in the time sequence image by determining an open position of the eyelid in the time sequence image, determining a closed position of the eyelid in the time sequence image, and identifying an eye blink according to the position of the eyelid relative to the open position and the closed position. The computer system may determine a blink completeness value of an eye blink of the one or more eye blinks.

The output includes the time sequence image, and the time sequence image has a blink overlay indicating an eye blink of one or more eye blinks.

The output includes the time sequence image, and the time sequence image has an eyelid overlay superimposed onto the eyelid shown in the column images.

The blink analysis output includes an eye opening graph that indicates the position of the eyelid during the imaging period.

The blink analysis output includes one or more of the following: a number of eye blinks during the imaging period; a blink rate representing a number of blinks over a unit of time; an average blink completeness representing a mean value of the blink completeness values of the one or more eye blinks; and/or a partial blink rate representing a number of partial blinks relative to the total number of blinks.

In certain embodiments, an ophthalmic system includes a computer system and a display. The computer system receives eye images of an eye that were obtained during an imaging period and generates a time sequence image from the eye images. The time sequence image indicates the position of an eyelid of the eye during the imaging period. The display displays a blink analysis output describing the position of the eyelid during the imaging period. The blink analysis output includes the time sequence image. The time sequence image includes column images in temporal order. Each column image has a respective longitudinal axis that is substantially parallel to a vertical axis of the eye. Each column image shows at least the eyelid at a respective time during the imaging period.

Embodiments may include one, more than one, any combination of, or all of the following:

The computer system generates the time sequence image from the eye images by extracting a column image from the each eye image to yield the column images and arranging the column images in temporal order to generate the time sequence image.

The ophthalmic system includes a camera system that can obtain the eye images of the eye during the imaging period.

The time sequence image has an eyelid overlay superimposed onto the eyelid shown in the column images.

The blink analysis output includes an eye opening graph indicating the position of the eyelid during the imaging period.

### BRIEF DESCRIPTION OF THE DRAWINGS

FIG. 1 illustrates an example of an ophthalmic system for analyzing the blink behavior of an eye, according to at least one embodiment described in the present disclosure;

FIG. 2 illustrates examples of applications that may be used to perform eye blink analyses for an eye, according to at least one embodiment described in the present disclosure;

FIG. 3 illustrates an example computing system, according to at least one embodiment described in the present disclosure;

FIGS. 4A to 4D illustrate examples of images that may be used and/or displayed to analyze the blink behavior of an eye, according to at least one embodiment described in the present disclosure; and

FIG. 5 illustrates an example of a method of analyzing the blink behavior of an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In known techniques of blink analysis, the blink behavior of an eye is recorded to yield a video of the eye. Medical personnel may inspect the video or image processing may be applied to the video to determine blink rate and completeness. However, these methods of analysis may be time-consuming and/or subject to error.

Certain embodiments of the present disclosure relate to capturing images of an eye and generating from the images a time sequence image that summarizes the blink behavior of the eye. In the embodiments, a column image is extracted from each image. The column image may be a vertical strip that shows at least the pupil center and an eyelid of the eye (and may show more of the eye). The time sequence image includes the column images in temporal order. The time sequence image summarizes the blink behavior of the eye by indicating the position of the eyelid over a time period.

Certain embodiments of the present disclosure may provide improvements over known iterations of blink analysis. For example, the embodiments may present essential information of blink behavior in an image that effectively summarizes the behavior. As another example, a computer may efficiently analyze the image to provide metrics that describe the blink behavior more quickly than analyzing a video of the blink behavior.

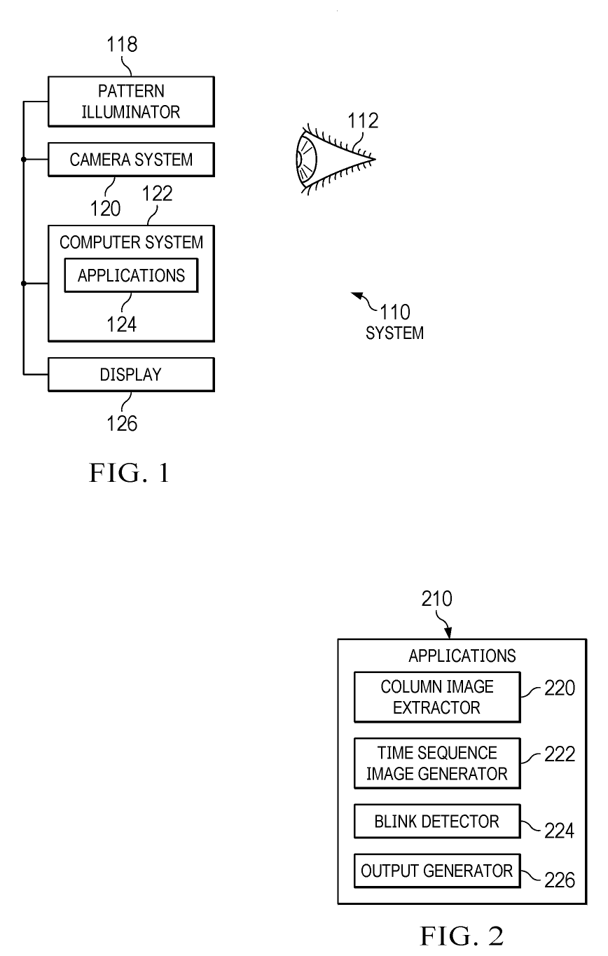

FIG. 1 illustrates an example of an ophthalmic system 110 for analyzing the blink behavior of an eye 112, according to at least one embodiment described in the present disclosure. In the example, the ophthalmic system 110 includes a pattern illuminator 118, a camera system 120, a computer system 122 (which may store computer applications 124), and a display 126, which may be coupled (communicatively or otherwise) as shown.

As an overview of the example, the camera system 120 obtains eye images of the eye 112 during an imaging period, and the computer system 122 receives the eye images from the camera system 120. The computer system 122 extracts a column image from each eye image. A column image has a respective longitudinal axis substantially parallel to the vertical axis of the eye 112 and shows at least the eyelid relative to the rest of the eye 112 along the longitudinal axis at a respective time during the imaging period. The computer system 122 generates a time sequence image comprising the column images in temporal order, which shows the position of the eyelid at different times during the imaging period. The computer system 122 outputs the time sequence image of the column images. In certain embodiments, the pattern illuminator 118 may direct a pattern of light towards the eye 112, and the camera system 120 may obtain images of the patterned light reflected from the eye. The computer system 122 may use the images to determine landmarks of the eye 112 (e.g., the pupil or other central region of the eye 112), which may provide guidance for where to extract the column image.

In general, the anterior portion of an eye 112 has a central region, e.g., a pupil center, a corneal apex, or a corneal vertex. A vertical axis (typically labeled the 90-270 degree-axis) may pass near or through the central region. The eye 112 also has an upper eyelid and a lower eyelid. The upper eyelid may close when the eye 112 blinks. Blinking is a normal reflex that protects the eye and regulates tears that nourish and cleanse the surface of the eye.

Turning to the example components of the ophthalmic system 110, the pattern illuminator 118 directs light in a pattern towards the eye 112. The pattern may be any suitable pattern that can be reflected from the eye 112 to detect features of the eye 112. For example, the pattern may include one or more of the following: (1) a pattern (e.g., circular, polygonal, or rectangular array) of dots, dashes, and/or other figures, of the same or different sizes, equally or unequally spaced; (2) a series of lines (e.g., solid, dotted, and/or dashed), of the same or different thicknesses, equally or unequally spaced; and/or (3) concentric rings (e.g., solid, dotted, and/or dashed), of the same or different thicknesses, equally or unequally spaced.

The pattern illuminator 118 may comprise any suitable arrangement of one or more light sources that can illuminate the eye 112. In certain embodiments, the pattern illuminator 118 includes one or more illuminator rings that can provide any suitable circular pattern of light. For example, the circular pattern may comprise dots, dashes, and/or other lines, or the circular pattern may be a solid circular line. Different illuminator rings may provide the same or different patterns. In some embodiments, the pattern illuminator 118 includes illuminator elements, such as pixel illuminators, where each pixel illuminates the eye 112 with a pixel of light. The computer system 122 may turn on and off particular pixel illuminators to yield a specific pattern.

The camera system 120 includes one or more cameras that can obtain images (e.g., individual images or a video) of the eye 112. A camera may be any suitable camera that can capture and/or record images, such as a digital camera that records the image as digital image data. A digital camera may include: an image sensor that detects light reflected from an object, such as a digital image sensor (e.g., charge-coupled device (CCD) or complementary metal-oxide-semiconductor (CMOS)); an image processor that converts the sensor output to digital image data representing the image; and a memory that records the image as image data.

The computer system 122 includes applications 124 that may extract column images from eye images, generate a time sequence image comprising the column images in temporal order, detect the blinking of the eye 112 in the time sequence image, and/or generate an output using the time sequence image. Examples of computer systems are described in more detail herein. The display 126 displays the output generated by the computer system 122.

FIG. 2 illustrates examples of applications 210 that may be used to perform eye blink analyses for an eye, according to at least one embodiment described in the present disclosure. Applications 210 may include, e.g., a column image extractor 220, a time sequence image generator 222, a blink detector 224, and/or an output generator 226. While illustrated as discrete applications, it will be appreciated that such a depiction is for convenience of conveying concepts of the present disclosure and is not limiting. For example, one or more of the applications 210 may be combined in a single logical process or routine, including for example, a single software program, a single programmed chip or electronic, or any other implementation.

In certain embodiments, the column image extractor 220 extracts column images from eye images. A column image has a respective longitudinal axis substantially parallel to (which may be coinciding with or separated from) the vertical axis of the eye. For ease of explanation, a column image may be regarded as comprising one or more vertical columns of pixels and a plurality of horizontal rows of pixels. The column image may have any suitable vertical length and any suitable horizontal width. For example, the vertical length may have any suitable length that, e.g., captures the eye between the top of the upper eyelid and the bottom of the lower eyelid when the eye is open. The horizontal width may be, e.g., sufficiently wide to allow a human to discern the upper eyelid in the image and sufficiently narrow to allow for multiple column images to be included in the time sequence image. For example, the width may be 1 to 10 pixels, e.g., 1, 1 to 3, 3 to 6, and/or 6 to 10 pixels, or may be 1 to 10 millimeters, e.g., 1 to 3, 3 to 6, and/or 6 to 10 millimeters. In certain embodiments, the horizontal width may be adjusted in response to the number of column images that are included in the time sequence image. For example, the horizontal width may be smaller for a larger number of column images, and the horizontal width may be larger for a smaller number of column images.

To extract a column image, the column image extractor 220 may detect the central region of the eye in at least one eye image. For example, the column image extractor 220 may detect the pupil center as the central region. As another example, the column image extractor 220 may detect the pupil, detect reflections of a centration light in the area of the pupil, and determine the central region from the reflections. As another example, the column image extractor 220 may detect the location of the uppermost part of the eyelid when it is open. The central region may be found below the uppermost part of the eyelid. After locating the central region, the column image extractor 220 extracts from each eye image a column image that includes at least a portion of the central region of the eye. In certain embodiments, the column image may be further processed. For example, the column image extractor 220 may apply a function (e.g., an averaging function such as a weighted averaging function) to the pixel values of a row to obtain a common pixel value and replace at least a subset or all of the pixel values of the row with the common pixel value.

The time sequence image generator 222 generates a time sequence image comprising the column images in a temporal order. In certain embodiments, the time sequence image generator 222 combines the column images in a temporal order in a direction perpendicular to the longitudinal axis of the column images. For example, the column images may be sized to the same number of pixels in height and/or width (such as a width of a single pixel). The column images may be stitched, concatenated, and/or otherwise combined together sequentially and/or progressively over time. As another example, the column images may be aligned based on a known feature, such as the bottom eyelid, the center of the pupil, or any other feature such that any minor variations in eye movement are accounted for as the time sequence image is generated.

The blink detector 224 detects the blinking of the eye in the time sequence image. In certain embodiments, the blink detector 224 detects one or more parts of the eye in the time sequence image and then detects blinks according to the detected parts. In the embodiments, the blink detector 224 may identify the pupil and/or the upper eyelid in the image to locate the position of the upper eyelid. For example, the blink detector 224 may detect an eyelid marker indicating the eyelid and then detect the position of the eyelid according to eyelid marker. An eyelid marker may be, e.g., a natural part of and/or an applied mark on the eyelid. As another example, the blink detector 224 may detect the pupil and then identify the upper border of the pupil between the pupil and the upper eyelid (the “pupil - upper eyelid border”). The blink detector 224 may then detect the position of the eyelid according to the pupil - upper eyelid border.

In certain embodiments, the blink detector 224 may determine an open position of the upper eyelid, a closed position of the upper eyelid, and/or a partially closed position of the upper eyelid in a time sequence image. An open position may be the maximum height along the vertical axis (typically labeled the 90-270 degree-axis) of the upper eyelid, a closed position may be the minimum height along the vertical axis, and a partially closed position may be between (e.g., 20 to 80 percent, such as 40 to 60 percent between) the open position and the closed position. The open position and the closed position may be determined in any suitable manner. For example, the open position may be calculated from a function (e.g., an average) of the locally maximum heights of the upper eyelid in the time sequence image, and the closed position may be calculated from a function (e.g., an average) locally minimum heights of the upper eyelid in the time sequence image. As another example, the open position and/or the closed position may be estimated from historical data of blinks of a comparable eye. As yet another example, the open position may be determined by asking the patient to open their eye 112 and then obtaining an image of the open eye and identifying the open position in the image of the open eye. Similarly, the closed position may be determined by asking the patient to close their eye 112, obtaining an image of the closed eye, and identifying the closed position in the image of the closed eye.

In certain embodiments, the blink detector 224 identifies one or more blinks in the time sequence image according to the position of the eyelid relative to the open position and/or the closed position of the eyelid. For example, the blink detector 224 may detect a blink if the upper eyelid moves towards the closed position beyond some threshold (e.g., at least 10, 25, or 50 percent towards the closed position) and then moves back towards the open position.

In certain embodiments, the blink detector 224 may determine a blink quality, such as blink completeness. A blink completeness value may measure the amount of closure of the eyelid over the eye. The blink detector 224 may detect a completed blink if the upper eyelid moves towards and reaches the closed position and then moves back towards the open position. The blink detector 224 may detect a partial blink if the upper eyelid moves to a partially closed position without reaching the closed position and then moves back towards the location of the open position. In an example, a blink completeness value of 100% may represent a completed blink, and a blink completeness value of less than 100% may represent a partial blink.

The blink detector 224 may count the number of blinks during the imaging period to determine a blink rate and may track the number of completed blinks and the number of partial blinks to determine a partial blink rate. A blink rate may represent the number of blinks over a period of time. For example, a completed blink rate may represent a relationship between the number of completed blinks and the total number of blinks, and a partial blink rate may represent a relationship between the number of partial blinks and the total number of blinks.

The output generator 226 generates a blink analysis output using the time sequence image. The blink analysis output describes the position of the eyelid during the imaging period and may have any suitable format. In certain embodiments, the blink analysis output may comprise the time sequence image in any suitable format. For example, the time sequence image may have an eyelid overlay superimposed onto the eyelid in the image. As another example, the time sequence image may have an open position overlay superimposed onto the open position of the eyelid in the image and/or may have a closed position overlay superimposed onto the closed position of the eyelid in the image. In certain embodiments, the blink analysis output may comprise an eyelid position graph indicating the position of the eyelid over the imaging period.

In certain embodiments, the blink analysis output may comprise one or more blink metrics, where a blink metric can describe a measurement of partial eye blinks, completed eye blinks, and/or other eye blinks or any combination of partial eye blinks, completed eye blinks and/or other eye blinks. Examples of blink metrics include a number of eye blinks, an eye blink rate, an average blink completeness, and/or a partial eye blink rate. The number of eye blinks may describe the number of blinks that occur during a time period (e.g., an imaging period or a subset of the imaging period). The eye blink rate may describe the number of blinks that occur during a unit of time. The average blink completeness may describe a mean value of the blink completeness values of a number of blinks. The partial blink rate may describe the number of partial blinks that occur relative to the total number of blinks.

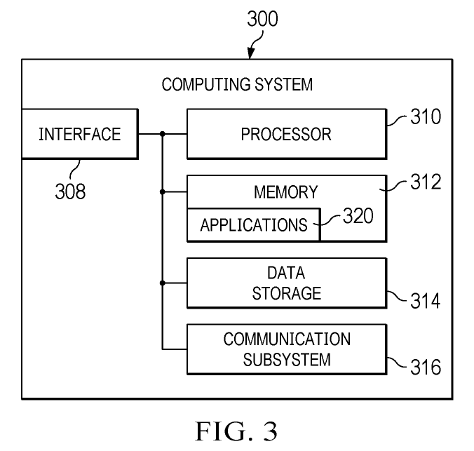

FIG. 3 illustrates an example computing system 300, according to at least one embodiment described in the present disclosure. The computing system 300 may include an interface 308, a processor 310, a memory 312, a data storage 314, and/or a communication subsystem 316, any or all of which may be communicatively coupled. The memory 312 may store one or more computer applications 320. Any or all of the computing system 300 may be implemented as computer hardware and/or software. Any or all of the systems disclosed herein may be implemented as a computing system consistent with the computing system 300.

In the example, the interface 308 may receive input to the computing system 300 and/or send output from the computing system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computing entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively. For example, the processor 310 may receive images of an eye, extract column images from the images, and generate a time sequence image from the column images.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310. For example, the memory 312 and/or the data storage 314 may store images of the eye, column images, and/or time sequence images.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

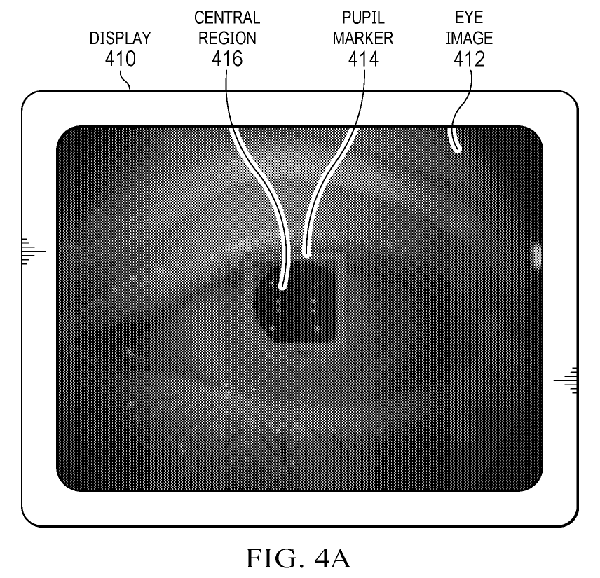

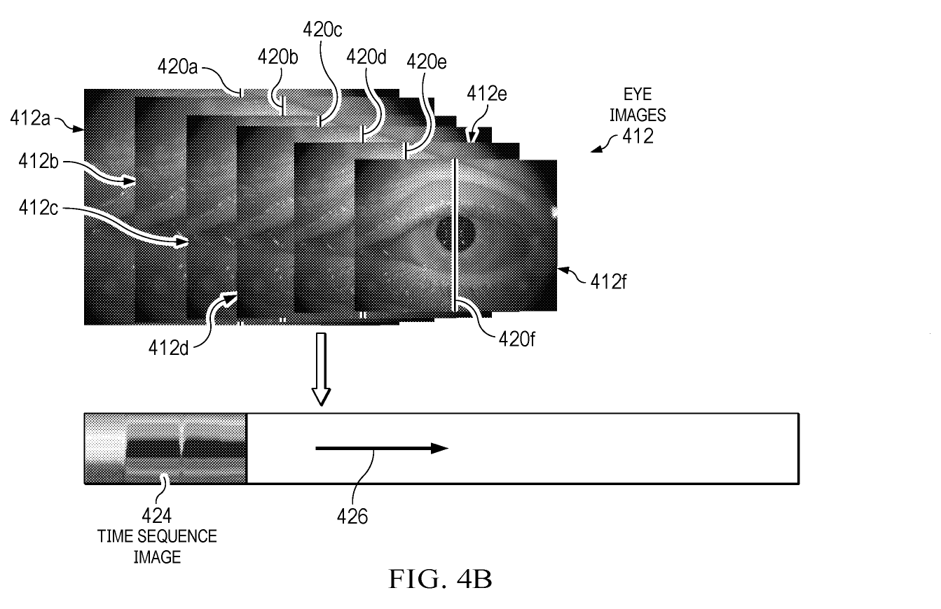

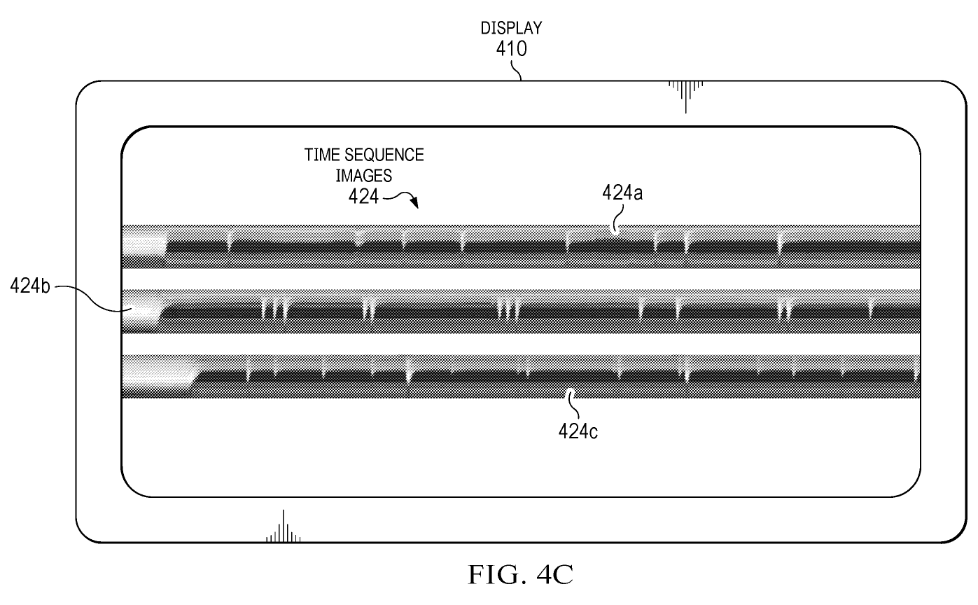

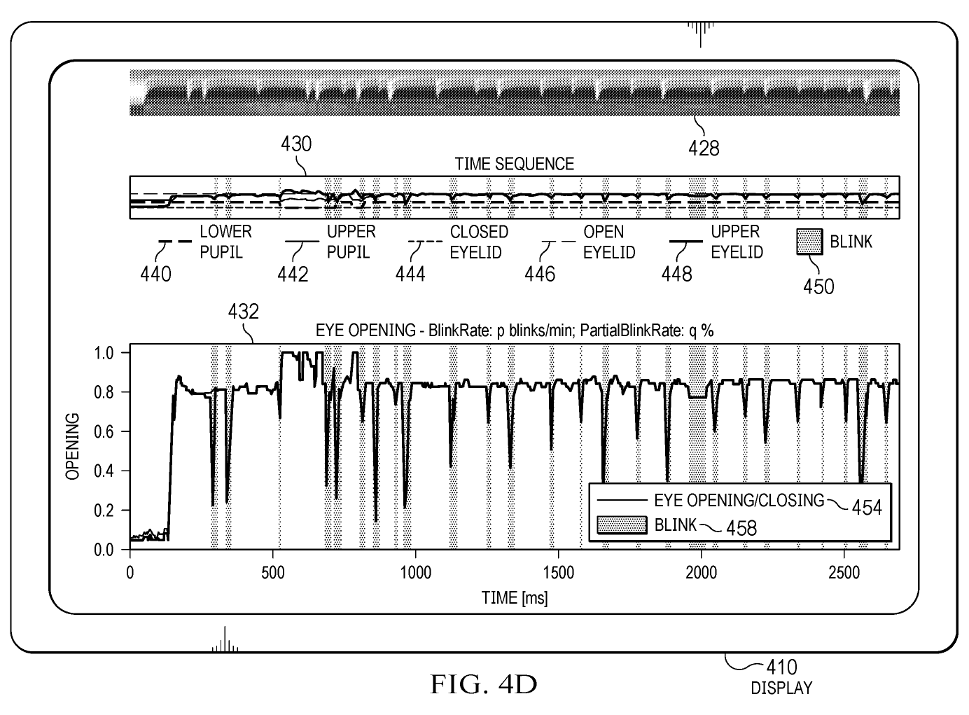

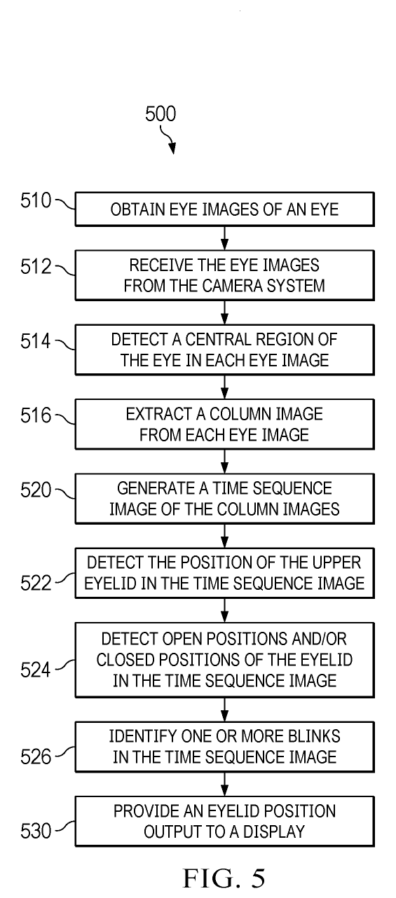

FIGS. 4A to 4D illustrate examples of images that may be used and/or displayed to analyze the blink behavior of an eye, according to at least one embodiment described in the present disclosure. FIG. 4A illustrates an eye image 412 presented by a display 410. The eye image 412 may include a pupil marker 414 indicating a central region 416 of the eye. The pupil marker 414 may have any suitable shape (e.g., a circle, square, polygon, or other suitable shape) and any suitable line pattern (e.g., solid, dashed, and/or dotted).

FIG. 4B illustrates multiple eye images 412 (412a to 412f) in temporal order from left to right. Each eye image 412 (412a to 412f) has a column image 420 (420a to 420f, respectively) that is extracted therefrom. The column images 420a to 420f are combined to form a time sequence image 424. The time sequence image 424 includes the column images 420 combined in temporal order in a direction 426 substantially perpendicular to the longitudinal axis of the column images 420. FIG. 4C illustrates time sequence images 424 (424a to 424c) presented by the display 410. Multiple time sequence images 424 may be taken of the same eye over different periods of time to yield multiple tests results, which may improve the accuracy of the analysis. For example, such analysis may be performed over three time segments, each of 2 seconds, 3 seconds, 4 seconds, 5 seconds, 10 seconds, 20 seconds, 30 seconds, 1 minute, or any other duration. As another example, one 5 second segment may be collected at a first visit and a second 5 second segment may be collected at a second visit. The analyses from the multiple visits may be compared, combined (e.g., averaged), or any other analysis performed on the multiple test results.

FIG. 4D illustrates examples of blink analysis outputs presented by the display 410. The blink analysis output includes a time sequence image 428, a time sequence image with overlay(s) 430, and an eye opening graph 432. The time sequence image 428 may be as described herein. The time sequence image with overlay(s) 430 may include one or more of the following overlays: a lower pupil overlay 440, an upper pupil overlay 442, a closed eyelid overlay 444, an open eyelid overlay 446, an upper eyelid overlay 448, a blink overlay 450, and/or other suitable overlay that indicates a part of the eye. An overlay may have any suitable shape (e.g., a circle, square, polygon, or other suitable shape), any suitable line pattern (e.g., solid, dashed, and/or dotted), and any suitable area pattern (e.g., shading, dots, and/or lines). The lower pupil overlay 440 indicates the boundary between the pupil and the lower eyelid, and the upper pupil overlay 442 indicates the boundary between the pupil and the upper eyelid, and such overlays may follow the position of the respective boundaries over time across the time sequence image with overlays 430. The closed eyelid overlay 444 indicates the closed position of an eyelid, the open eyelid overlay 446 indicates the open position of an eyelid, and such overlays may follow the position of the corresponding threshold over time across the time sequence image with overlays 430. The upper eyelid overlay 448 indicates the position of the upper eyelid, and the upper eyelid overlay 448 may include a trace that follows the position of the upper eyelid over time across the time sequence image with overlays 430. The blink overlay 450 highlights the detected blinks. In the example, the blink overlay 450 is shown as a shaded area that extends across the span representing the duration of the blink.

The eye opening graph 432 presents the range of motion of the pupil - upper eyelid border and/or the upper eyelid, which may indicate the completeness of blinks. In the example, the x-axis represents the time (in milliseconds), and the y-axis represents the amount the eye is opened or closed. In the example, 0.0 represents a closed eye, and 1.0 represents an open eye.

The eye opening graph 432 may include an eye opening / closing trace 454 and/or a blink overlay 458. The eye opening / closing trace 454 represents the position of the upper eyelid over time to show the opening and closing of the eye. The blink overlay 458 highlights the blinks. In the example, the blink overlay 458 is shown as a shaded area that extends across the span representing the duration of the blink.

In some embodiments, the eye opening graph 432 may include markers or visual indications of various thresholds. For example, a threshold may be displayed on the graph beyond which a blink (whether partial or full) is detected. As another example, a second threshold may be displayed beyond which a full blink is detected.

In some embodiments, one or more of the time sequence image 428, the time sequence image with overlays 430, and/or the eye opening graph 432 may be aligned temporally to facilitate viewing of one or more of the time sequence image 428, the time sequence image with overlays 430, and/or the eye opening graph 432. For example, the time sequence image 428, the time sequence image with overlays 430, and/or the eye opening graph 432 may be aligned vertically such that the blink overlay 450 of the time sequence image with overlays 430 may be disposed above the blink overlay 458 that represents the same blink.

In some embodiments, the eye opening graph 432 may display one or more blink metrics. In the example, the eye opening graph 432 displays the blink rate and/or partial blink rate calculated from the detected blinks. The blink rate represents the number of blinks per unit of time, and the partial blink rate represents the number of partial blinks over the total number of blinks. In the example, the blink rate is depicted as p blinks per minute, and the partial blink rate is depicted as q percent (e.g., the percentage of total blinks that are partial blinks).

While two example metrics are depicted (blink rate and partial blink rate), it will be appreciated that any number of metrics may be included in the display. For example, the metrics may include any of a number of partial eye blinks, a number of complete eye blinks, a number of eye total eye blinks, an eye blink rate, an average blink completeness, a partial eye blink rate, among others. In some embodiments, a user may select which metrics are to be displayed in conjunction with and/or instead of the time sequence image 428, the time sequence image with overlays 430, and/or the eye opening graph 432 as the output.

FIG. 5 illustrates an example of a method 500 of analyzing the blink behavior of an eye, according to at least one embodiment described in the present disclosure. The method 500 may be performed by any of the systems described herein.

At block 510, a camera system obtains eye images (e.g., eye images 412 (412a to 412f) of FIG. 4B) of an eye during an imaging period.

At block 512, a computer receives the eye images from the camera system.

At block 514, the computer detects a central region (e.g., central region 416 of FIG. 4A) of the eye in each eye image.

At block 516, the computer extracts a column image (e.g., column image 420 (420a to 420f) of FIG. 4B) from each eye image. A column image shows at least the eyelid of the eye at a respective time during the imaging period. A column image includes at least a portion of the central region of the eye and has a respective longitudinal axis substantially parallel to the vertical axis of the eye.

At block 520, the computer generates a time sequence image (e.g., time sequence image 424 of FIG. 4B) of the column images (e.g., column image 420 (420a to 420f) of FIG. 4B). The time sequence image comprises the column images in temporal order and indicates the position of the eyelid at different times during the imaging period.

At block 522, the computer detects the position of the upper eyelid in the time sequence image. The position of the eyelid may be detected in any suitable manner. For example, the computer may identify the pupil, the upper eyelid, and/or the pupil - upper eyelid border in the image to locate the position of the upper eyelid.

At block 524, the computer detects open positions and/or closed positions of the eyelid in the time sequence image. The open position of the eyelid occurs when the eye is open, and the closed position of the eyelid occurs when the eye is closed.

At block 526, the computer identifies one or more blinks in the time sequence image. The computer may detect a blink according to the position of the eyelid relative to the open positions and/or the closed position of the eyelid. Additionally or alternatively, the detection of blinks (whether partial or full) may be based on one or more thresholds. The computer may count the number of blinks (e.g., the number of completed blinks and/or partial blinks) during the imaging period to determine blink metrics (e.g., a number of eye blinks during the imaging period, an eye blink rate, and/or a partial blink rate).

At block 530, the computer provides a blink analysis output to a display. The blink analysis output describes the position of the upper eyelid, e.g., blinking, and may have any suitable format. In certain embodiments, the blink analysis output may comprise the time sequence image with and/or without overlays, an eye opening graph, and/or blink metrics. Examples of the blink analysis output are described herein, e.g., at FIGS. 4A through 4D.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. An ophthalmic system comprising:
a camera system configured to obtain a plurality of eye images of an eye during an imaging period, the eye having an eyelid; and
a computer system configured to:
receive the plurality of eye images of the eye from the camera system;
extract a column image from each eye image of the plurality of eye images to yield a plurality of column images, each column image of the plurality of column images having a respective longitudinal axis that is substantially parallel to a vertical axis of the eye, the each column image showing at least the eyelid of the eye at a respective time during the imaging period;
generate a time sequence image from the plurality of column images, the time sequence image comprising the plurality of column images in a temporal order, the time sequence image indicating a position of the eyelid of the eye at the respective times during the imaging period; and
output a blink analysis output, the blink analysis output comprising the time sequence image, one or more metrics associated with the time sequence image, or both the time sequence image and one or more metrics associated with the time sequence image.

2. The ophthalmic system of claim 1, the computer system configured to extract the column image from the each eye image of the plurality of eye images by:
detecting a central region of the eye in at least one eye image of the plurality of eye images; and
extracting the column image from the at least one eye image, the column image comprising at least a portion of the central region of the eye.

3. The ophthalmic system of claim 1, the computer system configured to detect the position of the eyelid in the time sequence image.

4. The ophthalmic system of claim 3, the computer system configured to detect the position of the eyelid in the time sequence image by:
detecting an eyelid marker of the eyelid of the eye in the time sequence image, the eyelid marker indicating the eyelid; and
detecting the position of the eyelid according to the eyelid marker of the eyelid of the eye.

5. The ophthalmic system of claim 3, the computer system configured to detect the position of the eyelid in the time sequence image by:
detecting a pupil of the eye in the time sequence image;
detecting an upper border between the pupil of the eye and the eyelid of the eye; and
detecting the position of the eyelid according to the upper border between the pupil of the eye and the eyelid of the eye.

6. The ophthalmic system of claim 1, the computer system configured to:
determine an open position of the eyelid in the time sequence image, the open position of the eyelid indicating the eye is open.

7. The ophthalmic system of claim 6, the computer system configured to:
provide an open eyelid overlay superimposed onto the open position of the eyelid in the time sequence image.

8. The ophthalmic system of claim 1, the computer system configured to:
determine a closed position of the eyelid in the time sequence image, the closed position of the eyelid indicating the eye is closed.

9. The ophthalmic system of claim 8, the computer system configured to:
provide a closed eyelid overlay superimposed onto the closed position of the eyelid in the time sequence image.

10. The ophthalmic system of claim 1, the computer system configured to identify one or more eye blinks in the time sequence image.

11. The ophthalmic system of claim 10, the computer system configured to identify one or more eye blinks in the time sequence image by:
determining an open position of the eyelid in the time sequence image;
determining a closed position of the eyelid in the time sequence image; and
identifying an eye blink of the one or more eye blinks according to the position of the eyelid relative to the open position of the eyelid and the closed position of the eyelid.

12. The ophthalmic system of claim 10, the computer system configured to:
determine a blink completeness value of an eye blink of the one or more eye blinks.

13. The ophthalmic system of claim 10, wherein the output includes the time sequence image, the time sequence image having a blink overlay indicating an eye blink of the one or more eye blinks.

14. The ophthalmic system of claim 1, wherein the output includes the time sequence image, the time sequence image having an eyelid overlay superimposed onto the eyelid shown in the plurality of column images.

15. The ophthalmic system of claim 1, the blink analysis output further comprises an eye opening graph indicating the position of the eyelid of the eye at the respective times during the imaging period.

16. The ophthalmic system of claim 1, the blink analysis output comprising one or more of the following:
a number of eye blinks during the imaging period;
a blink rate representing a number of blinks over a unit of time;
an average blink completeness representing a mean value of one or more blink completeness values of the one or more eye blinks; or
a partial blink rate representing a number of partial blinks relative to a total number of blinks.

17. An ophthalmic system comprising:
a computer system configured to:
receive a plurality of eye images of an eye, the plurality of images of the eye obtained during an imaging period; and
generate a time sequence image from the plurality of eye images of the eye, the time sequence image indicating a position of an eyelid of the eye at a respective time during the imaging period; and
a display configured to:
display a blink analysis output describing the position of the eyelid of the eye at the respective times during the imaging period, the blink analysis output comprising the time sequence image, the time sequence image comprising a plurality of column images, each column image of the plurality of column images having a respective longitudinal axis that is substantially parallel to a vertical axis of the eye, the each column image of the plurality of column images showing at least the eyelid of the eye at the respective time, the time sequence image comprising the plurality of column images in a temporal order.

18. The ophthalmic system of claim 17, the computer system configured to generate the time sequence image from the plurality of eye images of the eye by:
extracting a column image of the plurality of column images from each eye image of the plurality of eye images to yield the plurality of column images; and
arranging the plurality of column images in the temporal order to generate the time sequence image.

19. The ophthalmic system of claim 17, comprising a camera system configured to obtain the plurality of eye images of the eye during the imaging period.

20. The ophthalmic system of claim 17, the time sequence image having an eyelid overlay superimposed onto the eyelid shown in the plurality of column images.

21. The ophthalmic system of claim 17, the blink analysis output comprising an eye opening graph indicating the position of the eyelid of the eye at the respective times during the imaging period.
